(19) United States
(12) Patent Application Publication
(10) Pub. No.: US 2010/0063515 A1
(43) Pub. Date: Mar. 11, 2010

(54) ERGONOMIC NEEDLE WALDO AND METHOD

(75) Inventors: Frederick C. Fisher, Dakota Dunes, SD (US); Nathaniel J. Leon, Shawnee, KS (US)

Correspondence Address: SUITER SWANTZ PC LLO, 14301 FNB PARKWAY, SUITE 220, OMAHA, NE, 68154, US

(73) Assignee: Frederick C. Fisher, Dakota Dunes, SD (US)

(21) Appl. No.: 12/556,240

(22) Filed: Sep. 9, 2009

Related U.S. Application Data
(60) Provisional application No. 61/191,587, filed on Sep. 10, 2008.

(51) Int. Cl. A61B 19/00

(52) U.S. Cl. 606/130

(57) ABSTRACT

An ergonomic needle waldo may include a needle mounted in a needle head assembly coupled to a hinge assembly. The hinge assembly may be coupled to a tube body assembly and/or handle assembly. Additionally, a thumb wheel may be disposed between handle assembly and tube body assembly.

### CROSS-REFERENCE TO RELATED APPLICATIONS

The present application claims the benefit under 35 U.S.C. §119(e) of U.S. Provisional Application Ser. No. 61/191,587, filed Sep. 10, 2008, which is incorporated herein by reference.

### TECHNOLOGY FIELD

The present technology generally relates to the field of medical devices, and more particularly to an ergonomic needle waldo for fluoroscopic use.

### BACKGROUND

Fluoroscopy is an imaging technique often used by doctors and physicians to obtain real-time moving images of the internal structures of a patient through use of a fluoroscope. A simple fluoroscope may include an x-ray source and a fluorescent screen between which a patient may be placed. Often a continuous x-ray beam is passed through an examined body part and is transmitted to a monitor so the body part may be observed in detail.

Physicians may perform many different procedures using fluoroscopy, including barium x-rays, blood flow studies, orthopedic surgery, catheter insertion, and/or needle insertion, such as in back pain management. For example, physicians treating back pain may insert needles into the back locations where there is back irritation and pain for injecting analgesics and/or other medicaments.

### SUMMARY

An ergonomic needle waldo is described in accordance with exemplary embodiments of the present technology. In particular, the ergonomic needle waldo may include a needle mounted in a needle head assembly coupled to a hinge assembly. The hinge assembly may be coupled to a tube body assembly and/or handle assembly. Additionally, a thumb wheel may be disposed between handle assembly and tube body assembly. Further, the ergonomic needle waldo may include at least one side support.

It is to be understood that both the foregoing general description and the following detailed description are exemplary and explanatory only and are not necessarily restrictive of the technology as claimed. The accompanying drawings, which are incorporated in and constitute a part of the specification, illustrate an embodiment of the technology and together with the general description, serve to explain the principles of the technology.

### BRIEF DESCRIPTION OF THE DRAWINGS

The numerous advantages of the present technology may be better understood by those skilled in the art by reference to the accompanying figures in which:

FIG. 1 is an isometric view illustrating an ergonomic needle waldo in accordance with an exemplary embodiment of the present invention;

FIG. 2 is an isometric view of the ergonomic needle assembly illustrated in FIG. 1, wherein the needle head assembly is disconnected from the hinge assembly, and the hinge assembly is in a neutral position;

FIG. 3 is a plan view of the ergonomic needle assembly illustrated in FIG. 1, wherein the assembly is shown without the needle head assembly;

FIG. 4 is a cross-section view of the ergonomic fluoroscope needle illustrated in FIG. 1, wherein a drive system configured for controlling the rotation of the needle is shown;

FIG. 5 is a partial cross-sectional view of the ergonomic fluoroscope needle illustrated in FIG. 1, wherein a thumb wheel of the drive system is shown;

FIG. 6 is a partial cross-sectional view of the ergonomic needle illustrated in FIG. 1, wherein a drive system, a hinge assembly, and a portion of the needle head assembly is shown;

FIG. 7 is a partial isometric view of the ergonomic fluoroscope needle illustrated in FIG. 1, wherein the needle head assembly is coupled to the hinge assembly;

FIG. 8 is a partial isometric view of the ergonomic fluoroscope needle illustrated in FIG. 1, wherein the needle head assembly is shown detached from the hinge assembly;

FIG. 9 is an isometric view illustrating a simplified ergonomic fluoroscope needle, wherein the ergonomic fluoroscope needle is shown without an active hinge assembly;

FIG. 10 is a partial isometric view of the simplified ergonomic fluoroscope needle in FIG. 9, wherein the needle head assembly is shown; and

FIG. 11 is a partial isometric view of an ergonomic fluoroscope needle including two side supports.

### DETAILED DESCRIPTION

Reference will now be made in detail to embodiments of the technology, examples of which are illustrated in the accompanying drawings.

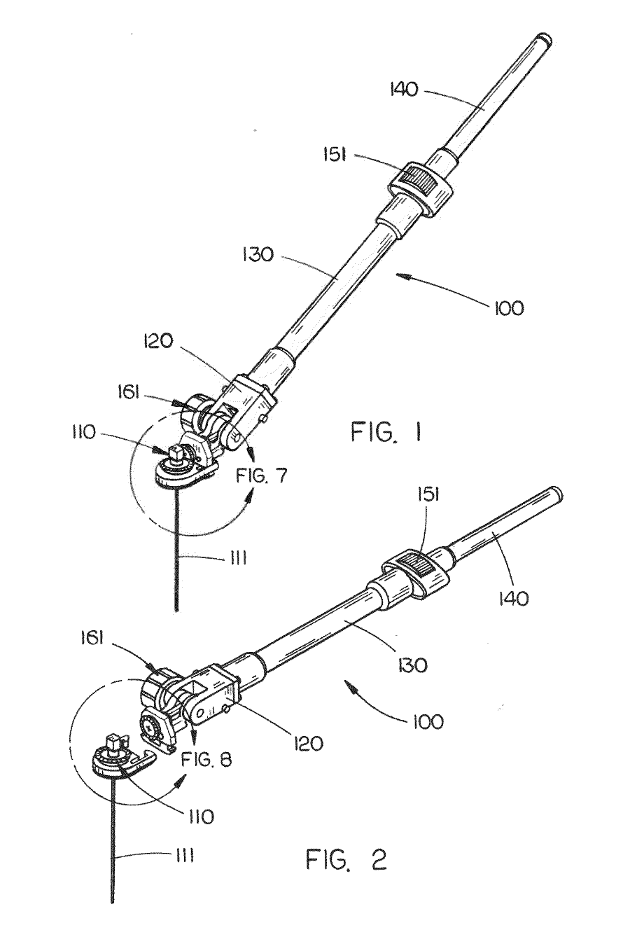

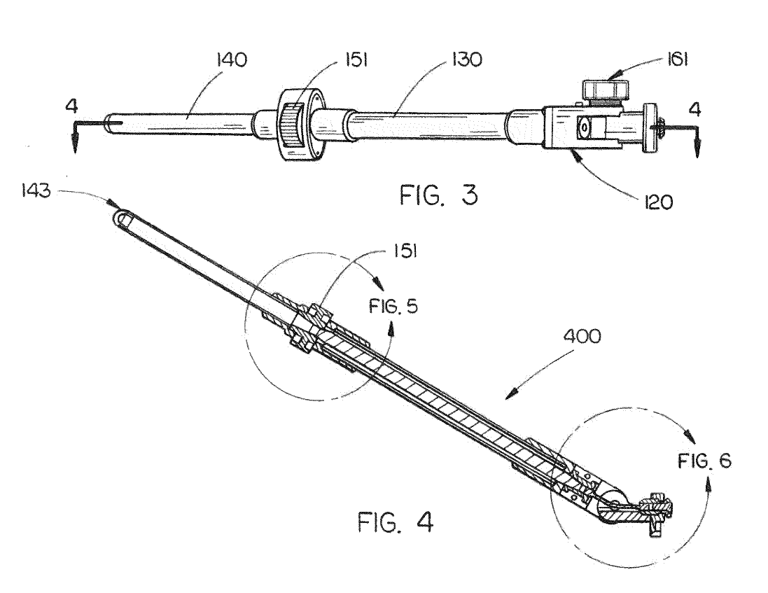

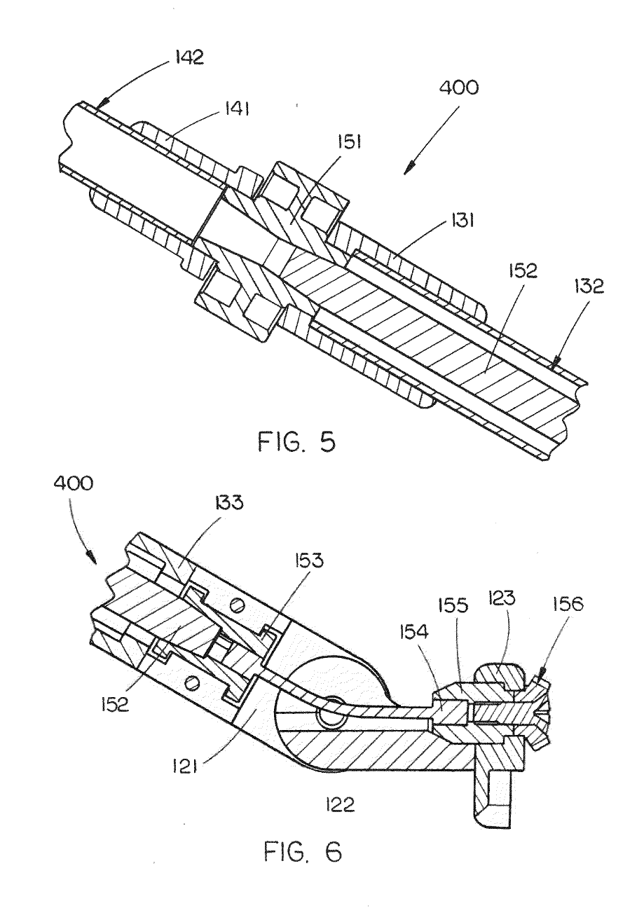

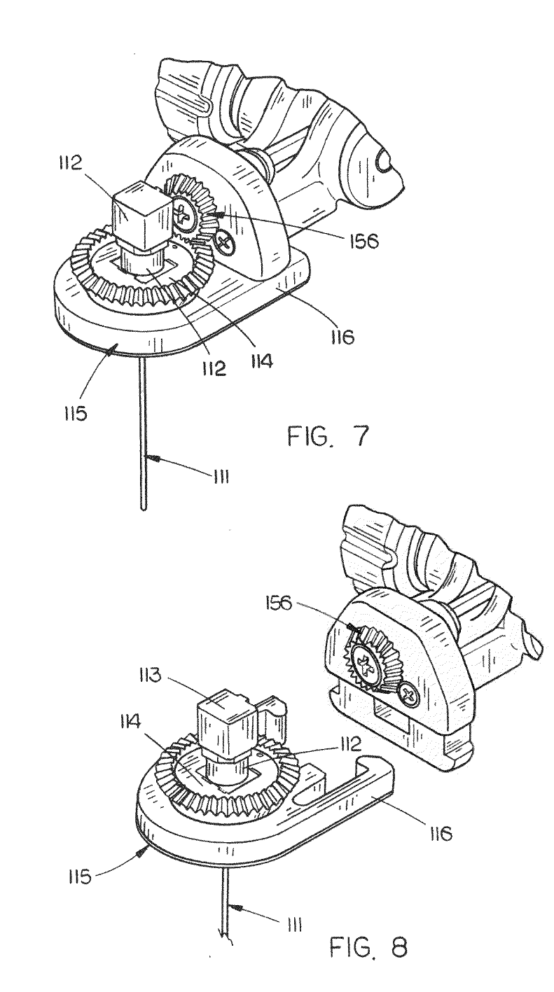

Referring generally to FIGS. 1 through 11, an ergonomic needle waldo 100 (or an ergonomic fluoroscope needle) is described in accordance with exemplary embodiments of the present technology. In particular, the ergonomic needle waldo 100 may include a needle 111 mounted in a needle head assembly 110 coupled to a hinge assembly 120. The hinge assembly 120 may be coupled to a tube body assembly 130 and/or a handle assembly 140. A thumb wheel 151 may be disposed between the handle assembly 140 and the tube body assembly 130. The thumb wheel 151 may connect to a drive system 400 that allows a user to rotate the needle 111 about its axis without exposure to an x-ray beam.

The ergonomic needle waldo 100 may include a hinge assembly 120. The hinge assembly 120 may allow the angle of the needle head assembly 110 to be adjusted to facilitate the positioning of the needle 111 and allowing the tip of the needle 111 to be moved. Further, the hinge assembly 120 may include a hinge locking nut 161 configured for loosening and/or locking the hinge assembly 120 so the user may adjust the angle between the needle head assembly 110 and the handle assembly 140.

The ergonomic needle waldo 100 may include a needle 111 suitable for medical use and may be fabricated primarily from radiolucent materials, for example acetal, phenolic, and/or sapphire radiolucent materials. A radiolucent material may include a material that permits the penetration and/or passage of x-rays. The needle 111, for example, may include a hypodermic needle. A hypodermic needle may include a hollow needle suitable for injecting a liquid into and/or sampling liquid from a body and a bevel edge on the hollow needle. The bevel edge may direct the general direction of an injected substance. In one embodiment, the ergonomic needle waldo 100 may include a hypodermic needle for injecting an antiseptic into a localized area of the body, such as an irritated area of a person's back. To ensure that a core of body material does not block the central aperture of the needle, a removable core wire 113 may be included. Additionally, the needle 111 may include a single-use needle and/or syringe. The needle 111 may range from a large gauge, such as 7 gauge, to a small gauge, such as a 33 gauge. In another embodiment, the ergonomic needle waldo 100 may include a 22 gauge single-use needle configured for injecting an antiseptic into a body. Further, the ergonomic needle waldo 100 may include a needle hub 112 configured for engaging and/or supporting the needle 111 and/or connecting it to a needle hub 112 or main gear 114 that may be used to assist rotation of the needle.

Additionally, at least a portion of the needle 111, the needle hub 112, main gear 114, or other portion of the ergonomic needle waldo 100 may include a radiodense portion for acting as a flag to indicate proper orientation of the needle 111 on a fluoroscope. Proper orientation of the needle 111 may include the orientation of the needle bevel for directing an injected material and/or guiding a needle 111 during an injection. One example of a radiodense material may include a metal. In an embodiment, a small portion of the needle hub may include a radiodense material, such as aluminum, stainless steel, and/or titanium.

The ergonomic needle waldo 100 may have two distinct degrees of freedom built into its structure. One degree of freedom may include the hinge assembly 120 configured for allowing the needle head assembly 110 to be positioned from inline to the handle assembly 140 to an angle in excess of 30 degrees. The second degree of freedom may include a drive system 400 configured to allow the needle 112 to be rotated about its central axis. In one embodiment, the needle 112 may be rotated continuously about its central axis.

In FIGS. 4 through 6, drive system 400 may be operated by a thumb wheel 151 located far enough from the Needle Head Assembly 110 to ensure that the user's hands are outside of the incident x-ray beam. The connection between the thumb wheel and needle may include a rigid drive Shaft 152, drive spool 153, flex shaft 154, coupler nut 155, and/or pinion gear 156. Pinion gear 156 may drive main bevel gear 114 when needle head assembly 110 is attached to the distal end of the hinge assembly 120. These components may be coupled, for example using shaft splines, threaded fasteners, mortise and tenon, or other mechanical attachments, so that thumb wheel 151 and needle 111 spin as one component. The ratio of the rotation of the thumb wheel 151 to the needle 111 may include many different ratios, such as 1:1, 2:1, or some other ratio as is normal with gears of this type.

Additionally, needle head assembly 110 may be detachable from hinge assembly 120 and/or the main body of this assembly and may be reused after sterilization or sanitation. The needle head assembly 110, including needle 111, may be disposed of by following standard medical protocols after a single use.

The ergonomic needle waldo 100 may be advantageous for several reasons. When inserting a needle into a patient while utilizing fluoroscopy, the doctor may be able to avoid direct exposure to x-ray beams from a fluoroscope. Additionally, the ergonomic needle waldo 100 may allow the doctor to control the angle and/or direction of the needle by using only one hand. Further, the ergonomic needle waldo 100 may allow the doctor to insert a needle into a patient's body without the need to resort to a point and shoot method. A point and shoot method may include inserting a needle part way into a body, taking a fluoroscope image, and then modifying the location of the needle. The point and shoot method takes extra time and may expose the patient to multiple needle insertions per injection and unnecessary movement of the needle causing discomfort to the patient.

One current method involves the use of a hemostat for holding a needle in place and while making an injection. However, utilizing a hemostat may cause the fluoroscope beam to auto-adjust to compensate for the metal mass of the hemostat. This compensation may cause the strength of the fluoroscope beam to increase and reduces the apparent contrast of the bones, cartilage, and needle tip. By utilizing an ergonomic needle waldo 100, this compensation may be avoided and x-ray exposure to the patient may be reduced. Additionally, the hemostat may not have the capability to rotate a needle relative to the hemostat when clamped for adjusting the direction of the bevel and/or directing the needle toward or away from the handle end of the hemostat.

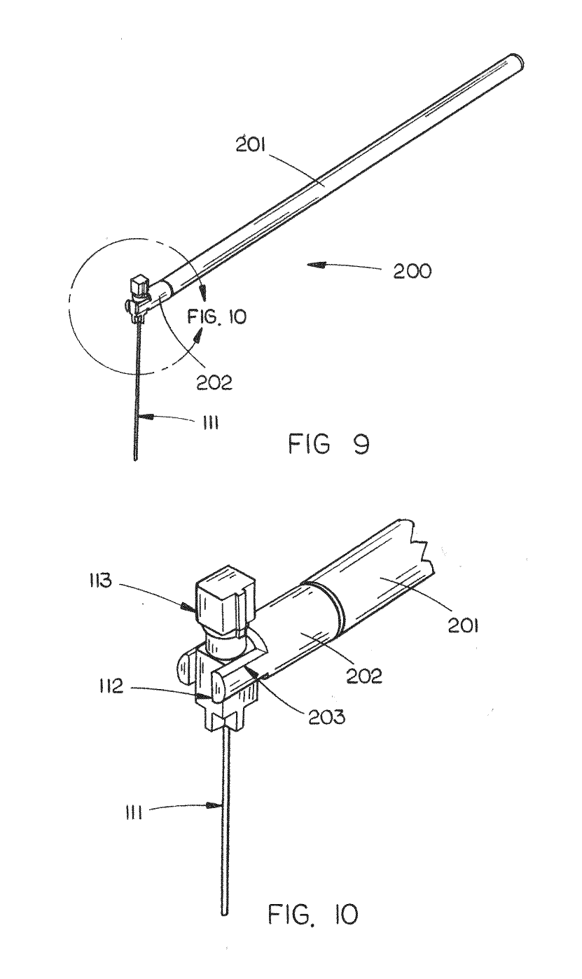

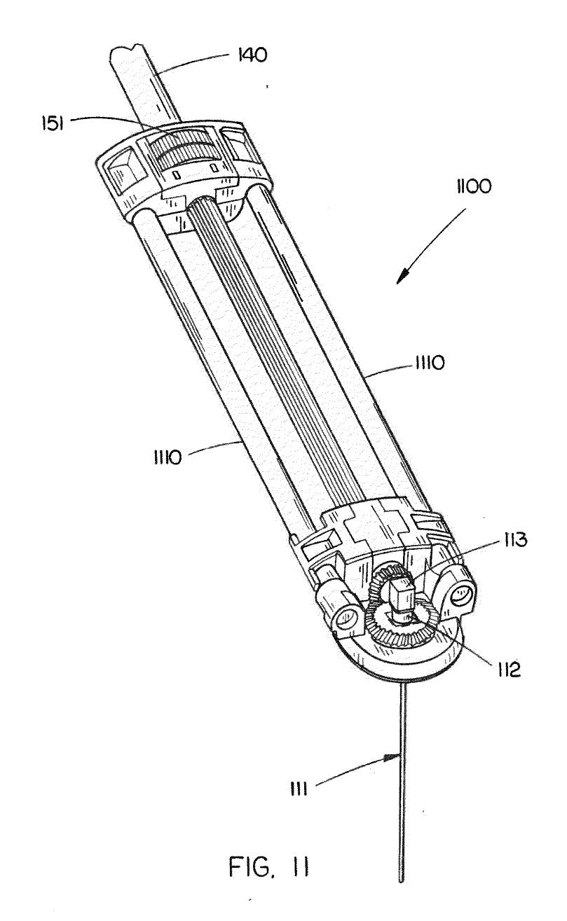

An alternate embodiment of this technology is shown in FIGS. 9 and 10. In this embodiment, the angle between shaft 201 and head 202 may be fixed at an angle configured to facilitate the insertion and direction of the needle tip toward the proximal end of the simplified fluoroscope needle assembly 200. The needle hub 112 may be fixed by clip tabs 203. The simplified fluoroscope needle assembly 200 may include radiolucent materials. This embodiment may be advantageous because the simplified fluoroscope needle assembly 200 may be inexpensive and may be disposed of after a single use.

As shown in FIG. 11 in an exemplary embodiment, an ergonomic fluoroscope needle 1100 may include at least one side support 1110. The at least one side support 1110 may extend parallel from needle hub 112 toward handle assembly 140 and/or thumb wheel 151. Each side support 1110 may comprise, for example, a suitable polymer, a composite, and/or metal material. In an embodiment, ergonomic fluoroscope needle 1100 may include two cylindrical shaped side supports 1110 extending from needle hub 112 toward handle assembly 140 and one side support 1110 disposed on each side of ergonomic fluoroscope needle 1100. When at least one side support 1110 is utilized, a user may be able to better control ergonomic fluoroscope needle 1100 for a longer period of time.

In the present disclosure, the methods disclosed may be implemented as sets of instructions or software readable by a device embodied in a tangible media, such as memory. Further, it is understood that the specific order or hierarchy of steps in the methods disclosed are examples of exemplary approaches. Based upon design preferences, it is understood that the specific order or hierarchy of steps in the method can be rearranged while remaining within the disclosed subject matter. The accompanying method claims present elements of the various steps in a sample order, and are not necessarily meant to be limited to the specific order or hierarchy presented.

It is believed that the present invention and many of its attendant advantages will be understood by the foregoing description, and it will be apparent that various changes may be made in the form, construction and arrangement of the components thereof without departing from the scope and spirit of the invention or without sacrificing all of its material advantages. The form herein before described being merely an explanatory embodiment thereof, it is the intention of the following claims to encompass and include such changes.

What is claimed is:

1. An ergonomic needle waldo, comprising:
a needle;
a needle hub configured for engaging the needle;
a needle adjustment assembly, including
the needle hub configured for engaging the needle, and
at least one gear configured to adjust the needle hub;
a tube body assembly coupled to the needle adjustment assembly; and
a handle assembly coupled to the tube body assembly.

2. The ergonomic fluoroscope needle in claim 1, further comprising:
a needle adjustment assembly, including
the needle hub,
at least one gear, and
means for adjusting the needle adjustment assembly.

3. The ergonomic fluoroscope needle in claim 1, comprising:
a fixed needle orientation.

4. The ergonomic fluoroscope needle in claim 1, comprising:
a hinge assembly coupled to the needle hub and the tube body assembly.

5. The ergonomic fluoroscope needle in claim 1, comprising:
a hinge locking nut.

6. The ergonomic fluoroscope needle in claim 1, comprising:
a thumb wheel configured to adjust a needle position.

7. The ergonomic fluoroscope needle in claim 1, comprising:
an ergonomic fluoroscope needle including a radiolucent material.

8. The ergonomic fluoroscope needle in claim 1, comprising:
a hypodermic needle.

9. The ergonomic fluoroscope needle in claim 8, comprising:
a hypodermic needle with a removable core wire.

10. The ergonomic fluoroscope needle in claim 1, comprising:
a radiodense portion.

11. The ergonomic fluoroscope needle in claim 1, comprising:
an ergonomic fluoroscope needle having at least two distinct degrees of freedom.

12. The ergonomic fluoroscope needle in claim 1, comprising:
a thumb wheel configured for rotating the needle.

13. The ergonomic fluoroscope needle in claim 1, comprising:
a thumb wheel coupled to the needle adjustment assembly by at least one of a rigid drive shaft, a drive spool, a flexible shaft, a coupler nut, or a pinion gear.

14. The ergonomic fluoroscope needle in claim 1, comprising:
a detachable needle head assembly.

15. The ergonomic fluoroscope needle in claim 1, comprising:
at least one side support.

16. The ergonomic fluoroscope needle in claim 1, comprising:
a hypodermic needle with a removable core wire;
a needle hub configured for engaging a detachable needle;
a needle adjustment assembly, including
the needle hub configured for engaging the needle, and
at least one gear configured to adjust the needle hub, where the at least one gear is configured to be adjusted by a thumb wheel;
a tube body assembly coupled to the needle adjustment assembly, where the tube body is configured to house means for adjusting the needle hub with a thumb wheel; and
a substantially flexible handle assembly coupled to the tube body assembly.
